(19) United States
(12) Patent Application Publication
(10) Pub. No.: US 2026/0069811 A1
(43) Pub. Date: Mar. 12, 2026

(54) INTEGRAL BITE BLOCK AND EXPANDABLE CHIN CUP OXYGEN MASK

(71) Applicants: Justin Robert Ibey, Highland, UT (US); Jerry Alan Ibey, Highland, UT (US)

(72) Inventors: Justin Robert Ibey, Highland, UT (US); Jerry Alan Ibey, Highland, UT (US)

(21) Appl. No.: 19/061,863

(22) Filed: Feb. 24, 2025

Related U.S. Application Data
(60) Provisional application No. 63/694,183, filed on Sep. 12, 2024.

(51) Int. Cl. A61M 16/06; A61M 16/04

(52) U.S. Cl. CPC A61M 16/0616; A61M 16/0493; A61M 16/0694

(57) ABSTRACT

An oxygen mask, typically of the kind used during medical procedures, is disclosed. The oxygen mask may include a nose portion, an expandable chin cup, and a bite block aperture. The mask may further include a bite block which may be slidably engaged with the bite block aperture. The oxygen mask provides a better alignment of the bite block with the wearer's mouth while also ensuring that the oxygen mask does not cause injury to the wearer's eyes by sliding on the wearer's face.

### CROSS-REFERENCE TO RELATED APPLICATIONS

This application claims the benefit of U.S. Provisional Application No. 63/694,183, filed Sep. 12, 2024, which is incorporated herein by reference in its entirety, including but not limited to those portions that specifically appear hereinafter, the incorporation by reference being made with the following exception: In the event that any portion of the above-referenced provisional application is inconsistent with this application, this application supersedes said above-referenced provisional application.

### BACKGROUND

### 1. Technical Field

This disclosure relates generally to an oxygen mask which includes a chin cup that is expandable and which includes an integral bite block for facilitating access to a patient's mouth and nose during administration of anesthesia.

### 2. Description of the Related Art

One aspect of human life is tolerating and recovering from injury or sickness. Throughout history, certain medicines and techniques were known to some cultures that could be used to help a hurt or sick person recover from their injury or sickness In some cases, a hurt or sick person needed another person to help perform a medical procedure to help the person recover. However, due to pain associated with the medical procedure, performing the medical procedure could be complicated. In many cases, remaining still is difficult for a person undergoing a procedure due to the pain and anxiety associated with the procedure. Early on, a need for anesthesia was recognized.

One of the earliest known anesthetics was the application of alcohol used to sedate a person experiencing a medical procedure. Other early anesthetics included opium from poppy plants. Some techniques known to ancient cultures have been lost, though some speculation exists as to the source of the anesthetics used by those cultures and how they were used. For most of human history, alcohol and opium were the main options for providing an anesthetic effect on a person, although some herbal alternatives were likely used. During the middle ages, it was discovered that cocoa leaves caused some anesthetic effect and became a new resource for use during medical procedures. Eventually, the extract of the cocoa leaf, cocaine, was isolated and came into use for anesthesia.

Anesthesia, as we know it today, is a relatively recent discovery. While ether and nitrous oxide were known in the 18^{th} and 19^{th} centuries, they were unpredictable and difficult to use in the correct amounts. The first known surgery performed under general anesthesia was not performed until 1804 in Japan while western countries did not conduct a successful anesthetic surgery until 1846. However, due to the volatility of both ether and nitrous oxide, the application of anesthesia was difficult to master. Later, when chloroform was discovered, it was also determined that a mask may be more advisable to keep the anesthesia contained and applied to the patient with minimal exposure to the person performing the medical procedure.

As technology improved, masks were developed to apply anesthetic and oxygen to a patient while also allowing carbon dioxide to be exhaled from the patient's body. Various masks have been innovated based on new surgical techniques or the needs of both physicians performing surgery and by the patient receiving the surgery. When anesthetized, using modern techniques, a patient may lose the ability to control their bodies. At the same time, an anesthesiologist may have a need to keep a patient's mouth open to intubate, extubate, or to prevent dental damage due to medical tools being inserted into the patient's mouth. Bite blocks were created to address these issues. A bite block is typically applied to a patient's mouth before anesthesia is administered to ensure that doctors can access the patient's airway during a medical procedure.

The bite block has large hole through a middle of the block, which provides access for medical tools into the mouth or throat of the patient. The bite block is positioned in a patient's teeth as a separate instrument to keep the patient's mouth open, which can be upsetting and uncomfortable for a patient prior to anesthesia. Worse, the bite block has a tendency of preventing a complete seal of a surgical mask on the patient's face. While some masks include holes which are intended to provide access through the mask to a bite block, since the mask and bite block are not connected, the mask tends to shift position undesirably when an oral scope, camera, or light is inserted into the bite block. The mask frequently is pushed into the patient's eyes or otherwise moved due to the physician trying to align an access hole in the mask to the hole in the bite block to insert a tool into the mouth of a patient.

Given this difficulty it is one object of this disclosure to provide a surgical mask which includes an integral bite block. Another object of this disclosure is to provide a surgical mask which provides an expandable chin cup.

### SUMMARY

Disclosed herein is an oxygen mask. The oxygen mask may include a nose portion, an expandable portion, and a bite block aperture. The oxygen mask may accept a bite block which may be installed in the bite block aperture. The expandable portion may include one or more folds which allows the mask to be selectively lengthened based on movement of the wearer's mouth, particularly for opening to accept the bite block into the wearer's mouth at an optimal angle.

### BRIEF DESCRIPTION OF THE DRAWINGS

The accompanying drawings illustrate various embodiments of an oxygen mask disclosed herein.

FIG. 1 illustrates an exemplary oxygen mask.

FIG. 2 illustrates the exemplary oxygen mask of FIG. 1 in an expanded state.

FIG. 3 illustrates another embodiment of an oxygen mask.

### DETAILED DESCRIPTION OF PREFERRED EMBODIMENTS

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

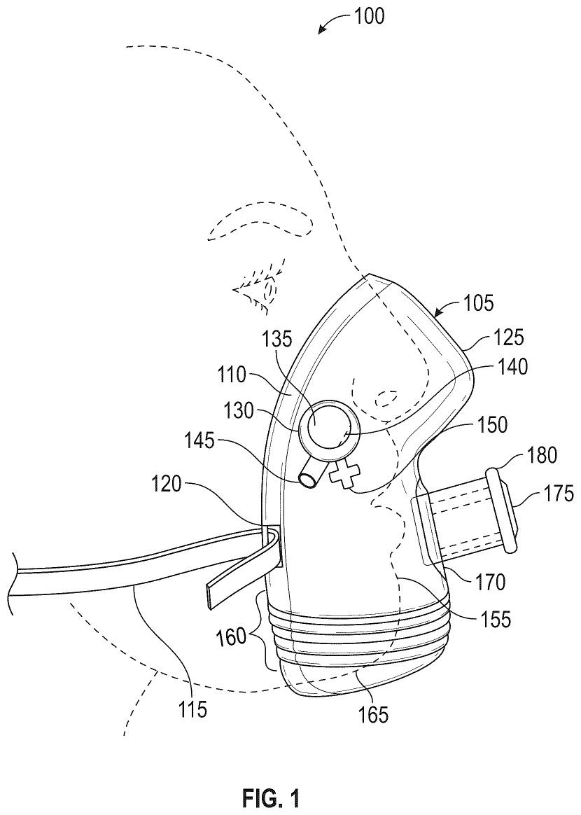

FIG. 1 illustrates an exemplary surgical mask device 100. Device 100 may be implemented as an oxygen mask 105 which is conventionally used for the administration of oxygen to a wearer and the removal of carbon dioxide from a wearer. Mask 105 may be constructed from any substance that can be sterilized. For example, preferable materials for constructing mask 105 may include plastics, metals, or metal alloys. In one embodiment, a plastic mask may be flexible and pliable mask, made from silicone, rubber, or other plastics with similar characteristics to silicone and rubber known in the art.

Mask 105 may be fitted with a seal 110 which may have a rounded edge to prevent discomfort from an edge with a corner. The seal 110 interacts with a wearer's face to provide anesthesia, oxygen, or any other gaseous substance to the wearer of the mask. Seal 110 may or may not be a so called “air-tight” seal which would prevent ambient air from entering mask 105 or prevent oxygen inside the mask from escaping through seal 110. Mask 105 may be secured over the mouth, nose, and chin of the wearer by a strap 115. Strap 115 extends around the wearer's head (or ears) and may be implemented with an elastic material to hold mask 105 securely on the wearer's face. Mask 105 may be fitted with connectors, such as connector 120 which may allow for a point at which strap 115 may connect to mask 105.

Mask 105 may generally be shaped to accommodate a wearer's face. For example, a nose portion 125 may be fitted into mask 105 to allow mask 105 to cover a wearer's nose and seal 110 to adequately close around the wearer's nose. Nose portion 125 of mask 105 may extend outwardly in a convex shape in mask 105 to accommodate a wearer's nose without coming into contact with the wearer's nose. Nose portion 125 may further be positioned at a top section of mask 105.

Mask 105 may further include an internal oxygen fitting 130. Oxygen fitting 130 may further include a first compartment 125 for allowing gasses to enter mask 105 and a second compartment 140 for allowing gasses to be removed from mask 105. First compartment 125 may be fitted with a hose connector 145 which may be connected to a hose that supplies oxygen or other gasses to mask 105. Second compartment 140 may further include a hose connector 150 which connects to a hose that removes carbon dioxide from mask 105. In a preferable embodiment, connector 145 and connector 150 may be disposed adjacent to each other for ease of access by a physician. Internal oxygen fitting 130 may be implemented in nose portion 125 of mask 105, in chin portion 155 (which will be discussed below) or between nose portion 125 and chin portion 155.

Mask 105 may further include an expandable portion 155 which includes an expandable portion 155. As shown in FIG. 1, expandable portion 155 is implemented by a number of folds or pleats 160 which allow mask 105 to expand and contract as the wearer's mouth is opened or closed. The folds or pleats 160 are merely exemplary and are disposed in an accordion like fashion where a plurality of folds are created in mask 105 that can extend or retract as desired. For example, when a wearer opens its mouth, folds 160 may allow expandable portion 155 to expand as the wearer's chin moves down to open the mouth. When the wearer closes its mouth, folds 160 may return to a folded position which operates to allow the mask to reduce size to still seal around the wearer's chin by chin cup 165. In this manner, chin cup 165 may be fitted under the chin of the wearer to secure mask 105 under the wearer's chin and maintain seal 110.

As the wearer opens its mouth, chin cup 165 allows pressure applied to chin cup 165 by a wearer's chin moving down to cause folds 160 to extend and allows chin cup 165 to maintain seal 110. In many cases, conventional masks experience displacement when a wearer opens its mouth. Here, chin cup 165 operates to prevent displacement of the mask by fitting chin cup 165 over the chin of a wearer, as shown in FIG. 1. It is also noted that folds or pleats 160 are only exemplary of embodiments of this invention. Other techniques that allow for mask 105 to be expandable are conceived. For example, a material selected for mask 105 may be naturally pliable or elastic and allow mask 105 to expand when experiencing pressure through chin cup 165. Other examples will be discussed below. However, this disclosure contemplates an expandable mask having a chin cup which responds to a wearer opening its mouth and closing its mouth.

Mask 105 may further include an aperture 170 for a bite block 180. Aperture 170 may simply be a hole through mask 105 or may include a frame installed in aperture 170 to provide strength to an area around aperture 170. For example, aperture 170 may be made of a thicker material around aperture 170, an inserted metal disc, or another type of plastic disposed around aperture 170 to provide rigidity and strength to aperture 170.

Bite block 180 may be integral to mask 105, may be inserted into mask 105 by aperture 170 or may be withdrawn from mask 105. Bite block 180 may be a single piece of a plastic, silicone, or rubber material. Bite block 180 may further incorporate a series of interlocking plastic components to extend or contract telescopically. Bite block 180 may serve to be inserted into a mask wearer's mouth to ensure that the wearer's mouth remains open. Bite block 180 may be a hard plastic or rubber material to prevent a wearer from crushing bite block 180 with its jaw or teeth. Bite block 180 may be fitted into aperture 170 and may include an optional retainer to prevent bite block 180 from being removed from mask 105. Bite block 180 may further be slidable through aperture 170 which allows bite block 180 to be inserted into the wearer's mouth and removed from the wearer's mouth, as desired. Bite block may further include a through-hole 175 which allows access to the wearer's mouth and airways during a procedure. For example, various scopes, cameras, lights, or other endoscopic tools may gain access to the mask wearer's mouth through bite block 180. Bite block 180 further prevents a wearer from biting down on the tools inserted into the wearer's mouth. Through-hole 175 may further incorporate a gaseous (or semi-permeable) membrane which tends to prevent gas inside mask 105 from mixing with gas outside 105 in an uncontrolled fashion. Advantageously, bite block 180 further operates as an anchor point for mask 105 to ensure that mask 105 does not shift position once installed over the wearer's mouth, nose, and chin.

The positioning of bite block 180 in mask 105 along with chin cup 165 provides a direct access with better alignment for a physician attempting to introduce an endoscopic tool, camera, scope, light, or other instrument into the wearer's esophagus or trachea than previous solutions. Mask 105 allows a more universal fit on all shapes and sizes of faces while also facilitating use in unusual circumstances, such as birth deformities and trauma to the facial area.

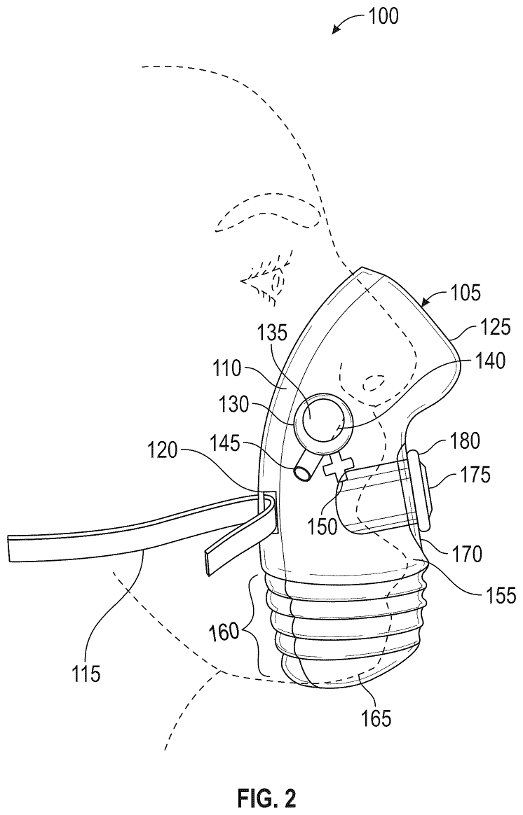

FIG. 2 illustrates the exemplary mask device 100 of FIG. 1 in an expanded state. Device 100 may be implemented as an oxygen mask 105 which is conventionally used for the administration of oxygen to a wearer and the removal of carbon dioxide from a wearer. Mask 105 may be constructed from any substance that can be sterilized. For example, preferable materials for constructing mask 105 may include plastics, metals, or metal alloys. In one embodiment, a plastic mask may be flexible and pliable mask, made from silicone, rubber, or other plastics with similar characteristics to silicone and rubber known in the art.

Mask 105 may be fitted with a seal 110 which may have a rounded edge to prevent discomfort from an edge with a corner. The seal 110 interacts with a wearer's face to provide anesthesia, oxygen, or any other gaseous substance to the wearer of the mask. Seal 110 may or may not be a so called “air-tight” seal which would prevent ambient air from entering mask 105 or prevent oxygen inside the mask from escaping through seal 110. Mask 105 may be secured over the mouth, nose, and chin of the wearer by a strap 115. Strap 115 extends around the wearer's head (or ears) and may be implemented with an elastic material to hold mask 105 securely on the wearer's face. Mask 105 may be fitted with connectors, such as connector 120 which may allow for a point at which strap 115 may connect to mask 105.

Mask 105 may generally be shaped to accommodate a wearer's face. For example, a nose portion 125 may be fitted into mask 105 to allow mask 105 to cover a wearer's nose and seal 110 to adequately close around the wearer's nose. Nose portion 125 of mask 105 may extend outwardly in a convex shape in mask 105 to accommodate a wearer's nose without coming into contact with the wearer's nose. Nose portion 125 may further be positioned at a top section of mask 105.

Mask 105 may further include an internal oxygen fitting 130. Oxygen fitting 130 may further include a first compartment 125 for allowing gasses to enter mask 105 and a second compartment 140 for allowing gasses to be removed from mask 105. First compartment 125 may be fitted with a hose connector 145 which may be connected to a hose that supplies oxygen or other gasses to mask 105. Second compartment 140 may further include a hose connector 150 which connects to a hose that removes carbon dioxide from mask 105. In a preferable embodiment, connector 145 and connector 150 may be disposed adjacent to each other for ease of access by a physician. Internal oxygen fitting 130 may be implemented in nose portion 125 of mask 105, in chin portion 155 (which will be discussed below) or between nose portion 125 and chin portion 155.

As previously mentioned, mask 105 may further include an expandable portion 155 which includes an expandable portion 155. Expandable portion 155 is implemented by a number of folds or pleats 160 which allow mask 105 to expand and contract as the wearer's mouth is opened or closed. The folds or pleats 160 are merely exemplary and are disposed in an accordion like fashion where a plurality of folds are created in mask 105 that can extend or retract as desired. For example, when a wearer opens its mouth, folds 160 may allow expandable portion 155 to expand as the wearer's chin moves down to open the mouth. When the wearer closes its mouth, folds 160 may return to a folded position which operates to allow the mask to reduce size to still seal around the wearer's chin by chin cup 165. In this manner, chin cup 165 may be fitted under the chin of the wearer to secure mask 105 under the wearer's chin and maintain seal 110.

As the wearer opens its mouth, chin cup 165 allows pressure applied to chin cup 165 by a wearer's chin moving down to cause folds 160 to extend and allows chin cup 165 to maintain seal 110. In many cases, conventional masks experience displacement when a wearer opens its mouth. Here, chin cup 165 operates to prevent displacement of the mask by fitting chin cup 165 over the chin of a wearer. It is also noted that folds or pleats 160 are only exemplary of embodiments of this invention. Other techniques that allow for mask 105 to be expandable are conceived. For example, a material selected for mask 105 may be naturally pliable or elastic and allow mask 105 to expand when experiencing pressure through chin cup 165. Other examples will be discussed below. However, this disclosure contemplates an expandable mask having a chin cup which responds to a wearer opening its mouth and closing its mouth.

As shown in FIG. 2, mask 105 is in an expanded state as folds 160 have been unfolded by the wearer opening its mouth. Chin cup 165 has been pushed down by the opening of the mouth which causes expandable portion 155 to lengthen, relative to FIG. 1. Since chin cup 165 rests on the underside of the wearer's chin, a simple mouth opening movement provides the force to expand folds 160 into the expanded state shown in FIG. 2. When the wearer closes the mouth, mask 105 may return to its normal state as shown in FIG. 1.

Mask 105 may further include an aperture 170 for a bite block 180. Aperture 170 may simply be a hole through mask 105 or may include a frame installed in aperture 170 to provide strength to an area around aperture 170. For example, aperture 170 may be made of a thicker material around aperture 170, an inserted metal disc, or another type of plastic disposed around aperture 170 to provide rigidity and strength to aperture 170.

Bite block 180 may be integral to mask 105, may be inserted into mask 105 by aperture 170 or may be withdrawn from mask 105. Bite block 180 may be a single piece of a plastic, silicone, or rubber material. Bite block 180 may further incorporate a series of interlocking plastic components to extend or contract telescopically. Bite block 180 may serve to be inserted into a mask wearer's mouth to ensure that the wearer's mouth remains open. Bite block 180 may be a hard plastic or rubber material to prevent a wearer from crushing bite block 180 with its jaw or teeth. Bite block 180 may be fitted into aperture 170 and may include an optional retainer to prevent bite block 180 from being removed from mask 105. Bite block 180 may further be slidable through aperture 170 which allows bite block 180 to be inserted into the wearer's mouth and removed from the wearer's mouth, as desired. Bite block may further include a through-hole 175 which allows access to the wearer's mouth and airways during a procedure. For example, various scopes, cameras, lights, or other endoscopic tools may gain access to the mask wearer's mouth through bite block 180. Bite block 180 further prevents a wearer from biting down on the tools inserted into the wearer's mouth. Through-hole 175 may further incorporate a gaseous membrane which tends to prevent gas inside mask 105 from mixing with gas outside 105 in an uncontrolled fashion.

Advantageously, bite block 180 further operates as an anchor point for mask 105 to ensure that mask 105 does not move once installed over the wearer's mouth, nose, and chin.

As shown in FIG. 2, bite block 180 has been positioned inside the wearer's mouth and such that a top portion of bite block 180 is positioned against mask 105. In this manner bite block 180 extends into the wearer's mouth and prevents the wearer's jaw or teeth from closing while bite block 180 is installed. In this manner, regardless of how the wearer's mouth is positioned, a physician has a direct alignment position to insert endoscopic tools into the wearer's mouth while also ensuring that the mask will not move. Not only does mask 105 enhance access to the mouth of the wearer but also increases efficiency of gas delivery to the patient. By providing a chin cup 165 and/or bite block 180, mask 105 is anchored which may prevent injury to the wearer in that mask 105 cannot slide into the wearer's eyes while wearing mask 105.

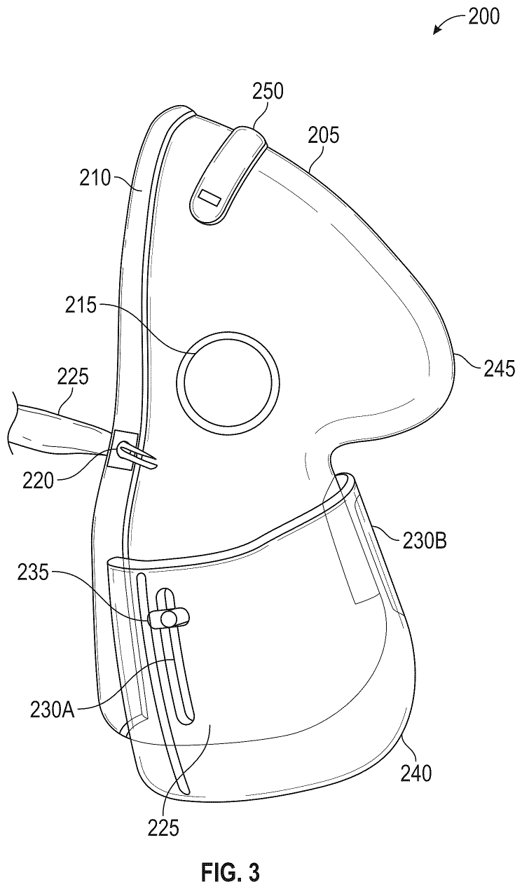

FIG. 3 illustrates another embodiment of a mask device 200. Mask device 200 may be implemented as, for example, an oxygen mask 205. Mask 205 may be constructed from any substance that can be sterilized. For example, preferable materials for constructing mask 205 may include plastics, metals, or metal alloys. In one embodiment, a plastic mask may be flexible and pliable mask, made from silicone, rubber, or other plastics with similar characteristics to silicone and rubber known in the art.

Mask 205 may be fitted with a seal 210 which may have a rounded edge to prevent discomfort from an edge with a corner. The seal 210 interacts with a wearer's face to provide anesthesia, oxygen, or any other gaseous substance to the wearer of the mask. Seal 210 may or may not be a so called “air-tight” seal which would prevent ambient air from entering mask 205 or prevent oxygen inside the mask from escaping through seal 210. Mask 210 may be secured over the mouth, nose, and chin of the wearer by a strap 225. Strap 115 extends around the wearer's head (or ears) and may be implemented with an elastic material to hold mask 205 securely on the wearer's face. Mask 105 may be fitted with connectors, such as connector 220 which may allow for a point at which strap 225 may connect to mask 205.

Mask 105 may further include an internal oxygen fitting 215 which may be implemented similarly to oxygen fitting 130, shown and described above with respect to FIG. 1. Oxygen fitting 230 may provide compartments and connections for external hoses to both introduce oxygen and other gasses while removing carbon dioxide, as discussed above.

Mask 205 may generally be shaped to accommodate a wearer's face. For example, a nose portion 245 may be fitted into mask 205 to allow mask 205 to cover a wearer's nose and seal 210 to adequately close around the wearer's nose. Nose portion 245 of mask 205 may extend outwardly in a convex shape in mask 205 to accommodate a wearer's nose without coming into contact with the wearer's nose. Nose portion 245 may further be positioned at a top section of mask 205. Further, nose portion 245 may include a nose clip 250 which may provide additional rigidity to mask 205 and ensure mask 205 remains in place. Nose clip 250 may be a metal strap which is bendable over a bridge of the wearer's nose.

Mask 205 may further include an expandable portion 225. In FIG. 3, expandable portion 225 is illustrated as a slidable chin cup 240 which is slidably connected to mask 205. For example, a slit 235 may be positioned in mask 205 while a pin or button 230A connects mask 205 to expandable portion 225. (A corresponding slit is not shown in the side of mask 205 opposite of slit 235 with a corresponding pin 230B). Expandable portion 225 may thereby slide up and down along mask 205 to lengthen and shorten mask 205. For example, when chin cup 240 is installed under a chin 240 of a wearer, expandable portion 225 may selectively expand or contract based on the wearer opening or closing the wearer's mouth.

A membrane and a bite block may be installed within mask 105, as discussed above with respect to FIG. 1, which may be disposed under the expandable portion 225 in mask 205. The membrane and bite block may be installed in a manner that allows a physician direct and aligned access through the bite block to the wearer's mouth.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A device, comprising:
a mask, the mask including:
a nose portion,
an expandable portion which includes a chin cup, and
an aperture which positions a bite block in a wearer's mouth.

2. The device of claim 1, wherein the mask includes a seal.

3. The device of claim 1, wherein the mask includes a gas aperture.

4. The device of claim 3, wherein the gas aperture includes a first compartment and a first hose connection.

5. The device of claim 4, wherein the gas aperture includes a second compartment and a second hose connection.

6. The device of claim 1, wherein the expandable portion selectively lengthens the mask.

7. The device of claim 1, wherein the expandable portion includes one or more folds.

8. The device of claim 1, wherein the chin cup is pinned to the mask and slides in one or more slits to selectively lengthen the mask.

9. The device of claim 1, wherein the bite block is installed in the aperture.

10. The device of claim 9, wherein the bite block includes a through-hole.

11. The device of claim 1, wherein the bite block is slidable through the aperture in the mask.

12. A mask device, the device including:
a nose portion;
a bite block, and
an expandable chin cup.

13. The device of claim 12, wherein the bite block is disposed in an aperture of the mask.

14. The device of claim 12, wherein the bite block is slidable in and out of an aperture of the mask.

15. The device of claim 12, wherein the bite block includes a through-hole.

16. The device of claim 12, wherein the expandable chin cup is disposed in an expandable portion of the mask.

17. The device of claim 16, wherein the expandable chin cup includes a plurality of folds.

18. The device of claim 17, wherein the plurality of folds are selectively unfolded by pressure applied by a chin on the chin cup to lengthen the mask.

19. The device of claim 12, further comprising an elastic strap.

20. The device of claim 19, wherein the elastic strap connects to the mask by one or more connectors.
